(19) United States
(12) Patent Application Publication
(10) Pub. No.: US 2023/0181475 A1
(43) Pub. Date: Jun. 15, 2023

(54) COMPOSITION COMPRISING METHYLFOLATE

(71) Applicant: ALFASIGMA S.P.A., Bologna (IT)

(72) Inventors: Mauro Marzi, Pomezia (RM) (IT); Mose' Santaniello, Pomezia (RM) (IT); Pietro Meloni, Pomezia (RM) (IT); Marta Paris, Pomezia (RM) (IT); Fabrizio Giorgi, Pomezia (RM) (IT); Emilio Merlo Pich, Bologna (IT)

(73) Assignee: ALFASIGMA S.P.A., Bologna (IT)

(21) Appl. No.: 17/998,534

(22) PCT Filed: May 10, 2021
(86) PCT No.: PCT/EP2021/062374

§ 371 Date: Nov. 11, 2022

(30) Foreign Application Priority Data
May 15, 2020 (IT) 102020000010741

(51) Int. Cl. A61K 9/20; A61K 31/519; A61K 9/16; A61P 25/24; A61K 45/06; A61K 31/205; A61K 31/675; A61K 31/714; A61K 9/28

(52) U.S. Cl. CPC A61K 9/2077; A61K 31/519; A61K 9/1652; A61K 9/2054; A61K 9/2059; A61K 9/1635; A61K 9/2027; A61K 9/1617; A61K 9/2009; A61K 9/2095; A61P 25/24; A61K 45/06; A61K 31/205; A61K 31/675; A61K 31/714; A61K 9/284

(57) ABSTRACT

The present invention describes a pharmaceutical and/or nutritional composition comprising methylfolate in the form of granules, together with a carnitine derivative salt, pharmaceutically acceptable excipients, and optionally other pharmaceutical or nutraceutical active ingredients. The composition is useful for oral administration. The invention also relates to the process for obtaining the composition comprising methylfolate in the form of granules and the use thereof for the treatment of disorders associated with a reduction of methylfolate, wherein methylfolate is useful.

### FIELD OF THE INVENTION

The present invention describes a pharmaceutical and/or nutritional composition comprising granules comprising methylfolate, together with granules comprising a carnitine derivative salt, pharmaceutically acceptable excipients, and optionally other pharmaceutical or nutraceutical active ingredients. The composition is useful for oral administration, preferably in form of tablet or sachet.

The invention also relates to the process for obtaining the composition comprising methylfolate in the form of granules and the use thereof for the treatment of disorders associated with a reduction of methylfolate, wherein methylfolate is useful.

### STATE OF THE ART

L-5-methylfolate is the metabolically active form of folic acid (vitamin B9) and it is able, through the transfer of a methyl group, to convert homocysteine back to methionine even in the presence of a genetic deficiency.

L-methylfolate or 6 (S)-5-methyltetrahydrofolate [6 (S)-5-MTHF] is the main biologically active diastereoisomer of folate and the primary form of folate in circulation. It is also the form that is transported through the membranes into the peripheral tissues, namely through the blood-brain barrier. In the cell, 6 (S)-5-MTHF is used in the methylation of homocysteine to form methionine and tetrahydrofolate (THF). THF is the immediate acceptor of a carbon unit for the synthesis of thymidine-DNA, purines (RNA and DNA) and methionine. About 70% of food folate and cell folate consists of 6 (S)-5-MTHF. Folic acid, the synthetic form of folate, must undergo an enzymatic reduction from methylenetetrahydrofolate reductase (MTHFR) to become biologically active. Genetic mutations of MTHFR determine the inability of a cell to convert folic acid into 6 (S)-5-MTHF. D-methylfolate or 6 (R)-5-methyltetrahydrofolate [6 (R)-5-MTHF] is the other diastereoisomer of folate. Studies that administered doses of 2.5 mg per day or more led to greater plasma protein binding of D-methylfolate than L-methylfolate, resulting in significantly higher renal clearance of L-methylfolate than D-methylfolate. In addition, D-methylfolate is stored in body' tissues, primarily in the liver. D-methylfolate is not metabolized by the body and has been postulated to inhibit regulatory enzymes.

The critical role of folate in brain metabolism and its metabolic pathways are known, and it has been noted that depressive symptoms are one of the most common neuropsychiatric manifestations in cases of folate deficiency. Patients with depression have folate levels in the blood which are, on average, 25% lower than in healthy controls, and low folate levels represent a strong predisposing factor to an unfavourable outcome of antidepressant therapy (Papakostas G. I. et al., Am J Psychiatry, 2012; 169 (12): 1267-74).

Other compounds can be useful in the treatment of disorders connected with depression and neuropathies, such as carnitine or derivatives thereof, and vitamins. Most folate receptors are found in the small intestine.

Methylfolate is also useful in the treatment of diabetic and peripheral neuropathies. Neuropathies cause numbness and sometimes pain and weakness in the hands, arms, feet and legs. These neurological problems can also occur in other organs, including the digestive tract, heart and sex organs. Diabetes patients can develop neuropathic problems at any time, but the more severe a person's diabetes, the greater the risk of developing said complications.

EP 2781214 describes a formulation comprising amorphous calcium L-5-methylyterahydrofolate and cysteine as stabilizing agent, wherein the formulation is prepared through a process comprising the step of blending MTHF and cysteine and forming the dosage form from the resulting blend. Said composition may comprise drosperidone and estradiol for use as contraceptive.

CN 107812195 describes a composition comprising MTHF with a reducing substance selected from vitamin C and its salt, isovitamin C and its salt, mercaptoethanol, cysteine, mercaptoethyl sulfonic acid, dithiothreitol, reduced glutathione, lipoic acid; the composition may further comprise estrogen and/or progesterone for use as contraceptive.

Monster Multi Dietary Supplement ae reported on http://www.gnpd.com is a commercial formulation comprising 20 vitamins and minerals for functional use on cardiovascular, bone health and immune system. MTHF and L-carnitine are comprised in the list of several components included in capsules.

U.S. Pat. No. 6,441,168 describes four stable crystalline forms of the calcium salt of 5-methyl-(6R, S), -(6R)- and -(6S)-tetrahydrofolic acid, the preparation process thereof and the use thereof for the production of medicaments and food additives.

Also carnitine and carnitine derivatives are known for their beneficial effect in disease associated with depression and neurological diseases.

U.S. Pat. No. 4,346,107 describes the use of acetyl L-carnitine for the therapeutic treatment of patients with impaired brain metabolism, such as in states of senile and presenile psychomotor involution and in senile and presenile dementia.

U.S. Pat. No. 4,343,816 describes the use of acetyl L-carnitine for the therapeutic treatment of patients with peripheral vascular diseases such as Raynaud's disease.

WO 98/57629 describes the use of acetyl L-carnitine for the therapeutic treatment of young individuals suffering from mood disorders classifiable as dysthymia and depressive, irritable, cyclothymic personality or temperament, involving a definite abuse of psychotropic substances.

WO 03/066041 describes the use of acetyl L-carnitine for the therapeutic treatment of depression in non-demented geriatric subjects with major depressive disorder (NDG-MDD).

EP 0256999 describes the use of acetyl L-carnitine for the treatment of acute and/or chronic peripheral neuropathies.

EP 1171111 describes acetyl L-carnitine granules suitable for the preparation of tablets without degradation of the active ingredient and solves the problem of instability of carnitine and the derivatives thereof in the presence of small amounts of water.

Vitamins of group B are useful for the treatment and prevention of neuropathies associated with a deficiency of said vitamins but are also a valuable aid in the case of non-deficient neuropathies, due to their analgesic, neuroprotective and anti-inflammatory effect. The B vitamins, in particular B6 (pyridoxine) and B12 (cobalamin), have an analgesic action, especially when taken in combination, due to the greater availability and/or efficacy of norepinephrine and 5-hydroxytryptamine, neurotransmitters that exercise an inhibitory action in the transmission of nociceptive pain. Said vitamins specifically inhibit some pathophysiological processes involved in neuropathic pain with a dose-dependent analgesic effect; higher doses correspond to more immediate and sustained benefits on pain symptoms. Finally, the B vitamins are an important therapeutic option in the treatment of peripheral neuropathies due to their neuroprotective action: they participate in numerous cellular metabolic reactions such as amino acid metabolism (vitamin B6), synthesis and regulation of DNA and fatty acids, energy production and folate methylation (vitamin B12); vitamin B12 also stimulates myelination processes, participating in the replacement of phosphatides, the main constituents of myelin fibre.

Folates, in particular the calcium salt, are used and marketed for all uses where there is a deficiency of said compound in the body, such as neuropathies and depressive states, but their stability is influenced by various environmental conditions such as changes in pH, temperature, oxygen or exposure to light.

There was also a need for pharmaceutical or nutritional compositions comprising methylfolate together with active ingredients, such as carnitine or derivatives thereof, vitamins, etc., for the treatment of depressive and neuropathic syndromes stable over the time without any degradation of MTHF. In the case of the latter, it was also useful for them to include vitamins such as vitamin B6 and vitamin B12.

The problem of the stability of 5-methyltetrahydrofolate is particularly relevant in the production and storage of pharmaceutical and nutraceutical compositions and also comprising other active ingredients which can influence the stability of the final composition. MTHF is unstable in various environmental conditions such as changes in pH, temperature, oxygen or exposure to light, which makes integration with food systems difficult.

Given the instability of methylfolate and its strong tendency to degrade, it was therefore necessary to have a composition comprising methylfolate which is stable over time. It was therefore important for the MTHF in said composition not to be degraded also by the other ingredients of the composition, such as salts and/or hydrated ingredients. To solve the problem of poor stability, Švarc P. L et al. in J. of Food Eng. 277, 2020, 109901 encapsulated MTHF by an electrospray process in the presence of biopolymers.

Liu Y. et al. in J. Agric. Food Chem. 2013, 61, 1, 247-254 solve the problem of MTHF stability by means of an ascorbate micro-encapsulation technique.

It was necessary to obtain stable, industrially scalable preparations of MTHF to be included in pharmaceutical or nutraceutical compositions, obtainable by reproducible processes, which were useful for all individuals with a folate deficiency and all individuals in whom methylfolate has a beneficial action.

It was also necessary to find a process for the production of pharmaceutical or nutraceutical compositions comprising L-5-methyltetrahydrofolate in combination with other active ingredients that maintained the stability of the finished product.

It has been found, and it is object of the present invention, a stable composition comprising granules comprising methylfolate, with granules comprising a carnitine derivative salt, preferably an acetyl L-carnitine salt, together with pharmaceutically acceptable excipients and optionally together with other natural or synthetic active ingredients, has been found and is the subject of the present invention.

The composition comprises granules comprising calcium L-methylfolate (MTHF) in crystalline form in an amount of 5 to 40% (w/w) with granules comprising a carnitine derivative salt, preferably an acetyl L-carnitine salt in an amount from 50 to 90% based on the weight of the finished composition, together with pharmaceutically acceptable excipients.

The composition is stable at 25° C. for at least 6 months, without any degradation of MTHF.

The methylfolate granules comprised in the final composition may be characterized by comprising an amount of methylfolate ranging from 2 to 10% by weight of the weight of the granulate.

### SUMMARY OF THE INVENTION

The present invention relates to a solid composition comprising granules comprising calcium L-methylfolate (MTHF) in crystalline form in an amount from 5 to 40% (w/w) on the weight of the finished composition, preferably with an antioxidant or stabilizer in an amount of 3 to 30% (w/w) with respect to the weight of the granules, and granules comprising an acetyl L-carnitine salt in an amount of 50 to 90% (w/w) on the weight of the finished composition, together with pharmaceutically acceptable excipients and optionally with other pharmaceutical or nutraceutical active ingredients.

The methylfolate granules may be characterized by comprising an antioxidant or a stabilizer in a methylfolate/antioxidant weight ratio from 1:1 to 1:20.

The invention describes a process for the preparation of the composition according to the invention, comprising the steps of:

a) preparation of granules comprising MTHF comprising mixing of MTHF in an amount of 3 to 10% (w/w) with an amount of 3 to 30%, preferably 3 to 15%, (w/w) of antioxidant or stabilizer, and optionally an amount from 50 to 90% (w/w) of diluent and an amount of from 4 to 10% (w/w) of binder, with respect to the weight of the granulate, followed by dry or wet granulation;

b) preparation of granules comprising a carnitine derivative salt, preferably an acetyl L-carnitine salt, comprising granulation of 75 to 90% (w/w) a carnitine derivative salt, preferably L-acetyl carnitine hydrochloride, optionally with 5 to 10% (w/w) microcrystalline cellulose and 5 to 10% (w/w) polyvinylpyrrolidone with respect to the weight of the granulate;

c) mixing the granules comprising MTHF obtained according to step a) with the granules of granules comprising a carnitine derivative salt, preferably a salt of acetyl-L-carnitine obtained according to step b) with the extragranular excipients; and optionally

d) compressing the mixture obtained in step c).

The composition according to the invention comprising MTHF granules is useful for all individuals in whom methylfolate is useful and has a beneficial effect, individuals with an insufficient dietary intake thereof, individuals with low values of L-methylfolate in the cerebrospinal fluid, plasma and/or blood, individuals with symptoms associated with depression in any form (mood disorder), bipolar disorder, cognitive disorders, psychotic disorders, schizophrenia and disorders connected with endothelial dysfunctions such as neuropathies, peripheral neuropathies or diabetic neuropathies.

### DESCRIPTION OF THE FIGURES

FIG. 1 reports the Immobility time (A) in FST on control (Ctrl) mice treated i.p. for 3 days with saline and on CUS mice treated i.p. for 3 days with saline, MF 3 mg/Kg, LAC 30 mg/Kg or LAC 30+MF 3 mg/Kg. After 3 days of treatment the time of immobility was significantly reduced in CUS mice by LAC 30 mg/Kg+MF 3 mg/Kg, n=8, means±SEM. * p<0.05 vs CUS Saline, CUS LAC 30 mg/Kg and CUS MF 3 mg/Kg in A, and vs Ctrl Saline in B. F (4,35)=10.95 in A, and F (4,35)=3,028 in B.

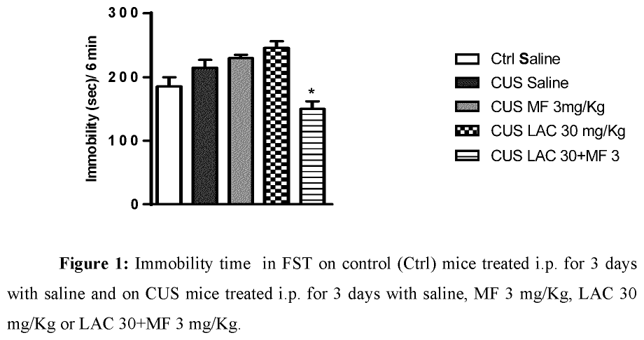

FIG. 2 reports Western blot analysis and representative blots of BDNF protein (mature form) in frontal cortex of Ctrl mice treated 14 days i.p. with saline. and CUS mice treated 14 days i.p. with saline. MF 3 mg/Kg. LAC 30 mg/Kg. or LAC 30+MF 3. n=2-6 mice per group. *p<0.05 vs all other groups. F (4.20)=7.459.

### DESCRIPTION OF THE INVENTION

The invention describes a solid composition comprising granules comprising calcium L-5-methyl-(6S)-tetrahydrofolate, also known as L-5-MTHF, L-methylfolate, L-5 methyltetrahydrofolate or (6S)-5-MTHF, L-5-Me-TH FA, L-5-Me-H4FA, L-5-Me-H4F, L-methylfolate and Metafolin®, hereinafter indicated also as methylfolate or MHTF or calcium methylfolate (hereinafter indicated also as MTHF granules), together with granules comprising a carnitine derivative salt (hereinafter indicated also as carnitine granules), and pharmaceutically acceptable excipients and, optionally other pharmaceutical or nutraceutical active ingredients.

Calcium L-5-methyl-(6S)-tetrahydrofolate (MTHF) comprised in the composition of the invention is in a crystalline form selected from Form I, Form II, Form III, Form IV, as described in U.S. Pat. No. 6,441,168, preferably in Form I.

The composition comprising MTHF in form of granules and a carnitine derivative salt in form of granules is useful for all individuals in whom the MTHF and the carnitine derivative are useful and have a beneficial effect. MTHF is useful in individuals with low values of L-methylfolate in the cerebrospinal fluid, plasma and/or blood, who have symptoms associated with depression, schizophrenia, cognitive disorders or psychotic disorders. The composition according to the invention is useful for the treatment and/or prevention of symptoms associated with depression, cognitive disorders or psychotic disorders.

MTHF is also useful for the treatment of disorders correlated with endothelial dysfunctions such as neuropathies, peripheral neuropathies and diabetic neuropathies. The composition according to the invention is useful for the treatment and/or prevention of disorders correlated with depression and/or schizophrenia, peripheral neuropathies and diabetic neuropathies.

The composition described is a pharmaceutical or nutraceutical composition or a food supplement.

The composition in solid form may be in the form of tablets, capsules, or granules for suspension, intended for oral administration.

The composition comprises MTHF in the form of granules in an amount ranging from 5 to 40% (w/w) of the weight of the final composition and acetyl L-carnitine in an of 50 to 90% in comparison to the total weight of the composition.

The MTHF granules are characterized by comprising an antioxidant and/or a stabilizing agent in a methylfolate to antioxidant/stabilizing agent weight ratio ranging from 1:1 to 1:20.

The MTHF granules comprise an amount ranging from 2 to 10% (w/w) of MTHF and an amount ranging from 3 to 30% (w/w) of a antioxidant and/or stabilizing agent together with pharmaceutically acceptable excipients.

The MTHF granules may comprise:
an antioxidant or stabilizing agent selected from the group comprising natural or synthetic agents or mixtures thereof. The natural agent is selected from the group comprising ascorbic acid, citric acid, resveratrol, vitamin E, carotenoids, coenzyme Q10, hydroxyacetophenone, cyclodextrins and sorbitol or mixtures thereof, preferably the antioxidant or stabilizing agent is selected from ascorbic acid, citric acid or mixture thereof. The synthetic agent is selected from the group comprising Captisol® (cyclodextrin), Vivapur® (carboxymethylcellulose and microcrystalline cellulose), or mixtures thereof. The granules may also comprise:
a diluent selected from the group comprising mannitol, corn starch, cellulose, microcrystalline cellulose, hydroxypropyl methylcellulose, lactose, sucrose, xylitol, sorbitol, dibasic calcium phosphate, calcium carbonate, kaolin, anhydrous or hydrated calcium sulphate, natural rubbers, malt, gelatin, or mixtures thereof;
a binding agent selected from the group comprising pregelatinized starch, cellulose, polyvinylpyrrolidone, gelatin, PEG, sucrose, sorbitol, cellulose derivatives, hydroxypropyl methylcellulose, gum arabic, copovidone, starch indicator, or mixtures thereof.

The MTHF granules may also comprise:
a buffering agent, selected from the group comprising phosphates, potassium or sodium acid phosphates, sodium or potassium hydroxide or mixtures thereof;
a chelating agent selected from the group comprising ethylenediaminetetraacetic acid sodium salt, citric acid, sorbitol, tartaric acid, phosphoric acid, or mixtures thereof;
a dehydrating agent selected from the group comprising microcrystalline cellulose, lactose, colloidal silica, kaolin, titanium oxide, alumina, sodium lauryl sulphate, aluminium and magnesium silicates, polyester and polyethylene or mixtures thereof.

In one aspect the MTHF granules comprise an amount ranging from 2 to 10% (w/w) of MTHF, an amount ranging from 3 to 30% (w/w) of an antioxidant and/or stabilizing agent, an amount ranging from 50% to 90% (w/w) of a diluent and an amount ranging from 2 to 10% (w/w) of binders, on the weight of the finished granulate.

The MTHF granules may comprise an amount ranging from 2 to 8% (w/w) of MTHF, 5 to 25% (w/w) of an antioxidant and/or stabilizing agent, preferably selected from ascorbic acid, citric acid or mixture thereof, 60 to 90% (w/w) of a diluent and 3 to 8% (w/w) of binders, on the weight of the finished granulate.

The MTHF granules comprise 2 to 8% (w/w) of MTHF, 5 to 25% (w/w) of citric acid or ascorbic acid or mixtures thereof, 60 to 90% (w/w) of corn starch or microcrystalline cellulose and 3 to 8% (w/w) of pregelatinized starch or hydroxypropyl cellulose, on the weight of the finished granulate.

In another aspect the granules comprise 2 to 8% (w/w) of MTHF, 5 to 15% (w/w) of ascorbic acid, 70 to 90% (w/w) of mannitol or modified starch and 2 to 6% (w/w) of hydroxypropyl cellulose, on the weight of the finished granulate.

In another aspect, the granules comprise 2 to 8% (w/w) of MTHF, 5 to 15% (w/w) of citric acid, 2 to 6% (w/w) of pregelatinized starch and 70% to 90% (w/w) of corn starch, on the weight of the finished granulate.

In another aspect, the granules comprise 3 to 8% (w/w) of MTHF, 5 to 15% (w/w) of ascorbic acid, 2 to 6% (w/w) of hydroxypropyl cellulose and 70 to 90% (w/w) of microcrystalline cellulose, on the weight of the finished granulate.

In a particular aspect, the granulate contains 30 grams of calcium methylfolate, 60 grams of ascorbic acid, 1200 grams of corn starch and 50 grams of pregelatinized starch.

The composition comprises MTHF granules in an amount ranging from 5 to 40% (w/w) and granules comprising a carnitine derivative salt in an amount ranging from 60 to 95% (w/w), on the weight of the finished composition.

The carnitine derivatives may be selected from acetyl L-carnitine, propionyl carnitine and the salts thereof selected from hydrochloride, fumarate, taurinate or mixtures thereof, preferably the carnitine derivative is acetyl L-carnitine, more preferably acetyl L-carnitine hydrochloride.

In one aspect, the carnitine derivative salts in the form of a granule.

In one aspect, acetyl L-carnitine hydrochloride in the form of granules with polyvinylpyrrolidone and microcrystalline cellulose is present in an amount ranging from 50 to 90% (w/w) on the weight of the finished composition.

In another aspect, the carnitine granule comprises acetyl L-carnitine hydrochloride in an amount ranging from 75 to 90% (w/w), carboxymethylcellulose in an amount ranging from 5 to 10% (w/w) and polyvinylpyrrolidone in an amount ranging from 5 to 10% (w/w) on the weight of the finished granulate. In a particular aspect, the acetyl L-carnitine granules are prepared according to EP 1171111.

In one aspect, the composition is in the form of tablets or granulate for oral administration.

The tablets may comprise MTHF granules in an amount ranging from 5 to 40% (w/w) and acetyl L-carnitine hydrochloride granules in an amount ranging from 50 to 95% (w/w), on the weight of the finished composition.

In one aspect, the composition in tablet form comprises MTHF granules in an amount ranging from 5 to 40% (w/w), acetyl L-carnitine, preferably hydrochloride, granules in an amount ranging from 50 to 90% (w/w), lubricant in an amount ranging from 0.1 to 15% (w/w), glidant in an amount ranging from 0.1 to 5% (w/w) and diluent in an amount ranging from 0 to 10% (w/w), on the weight of the tablet.

The MTHF granules and acetyl L-carnitine granules may be included in single-dose sachets wherein the dosage may be varied as required.

The extragranular excipients can be selected from disintegrants, glidants, lubricants and diluents, vitamins, other active ingredients or mixtures thereof.

The disintegrating agent is selected from the group comprising sodium starch glycolate, povidone (vinylpyrrolidone copolymer), crospovidone (polyvinylpyrrolidone/vinyl acetate copolymer), pregelatinized starch, sodium carboxymethyl cellulose (carmellose), crosslinked carboxymethyl cellulose (croscarmellose), sodium starch glycolate, calcium silicate, or mixtures thereof.

The lubricant is selected from the group comprising magnesium or calcium stearate, sodium stearyl fumarate, hydrogenated vegetable oils, mineral oils, polyethylene glycols, sodium lauryl sulphate, glycerides, sodium benzoate or mixtures thereof.

The glidant is selected from the group comprising talc, colloidal silica, precipitated silica, or mixtures thereof.

The tablet may comprise preservative, flavouring, colouring or sweetening agents, or mixtures thereof.

The tablet may be coated with a film coating which may optionally be a controlled-release coating.

In an aspect the unit composition in tablet form is as shown in Table 1.

TABLE 1

| Ingredient | % (w/w) |
|---|---|
| Acetyl L-carnitine granules | 60-80 |
| MTHF granules | 5-40 |
| Lubricant | 4-15 |
| Glidant | 1-5 |
| Diluent | 0-10 |

In one aspect, the tablet composition comprises MTHF granules ranging from 50 to 500 mg, acetyl L-carnitine hydrochloride granules ranging from 500 to 800 mg, and pharmaceutically acceptable excipients, useful for the tablet preparation.

In one aspect, the tablet composition comprises MTHF ranging from 2 to 30 mg, acetyl L-carnitine hydrochloride ranging from 400 to 700 mg, ascorbic acid or citric acid or mixture thereof ranging from 20 to 50 mg, lubricant ranging from 1 to 50 mg, glidant ranging from 0.1 to 10 mg, and diluent ranging from 0 to 100 mg, together with pharmaceutically useful excipients for the preparation of the tablet.

In a particular aspect the unit composition in tablet form is shown in Table 2.

TABLE 2

| | Ingredient | Amount (mg) |
|---|---|---|
| Acetyl L- | AcetylL-carnitine HCl | 590 |
| carnitine granule | | (=500 mg acetyl |
| | | L-carnitine) |
| | Microcrystalline | 35 |
| | cellulose | |
| | Polyvinylpyrrolidone | 56 |
| Methylfolate granule | MTHF | 15 |
| (GR6) | Ascorbic acid | 33 |
| | Corn starch | 172 |
| | Pregelatinized starch | 23 |
| Extragranular excipient | Magnesium stearate | 10 |
| Extragranular excipient | Colloidal silica | 3 |
| Film coating | Opadry AMB II | 40 |

In another aspect the unit composition in tablet form is shown in Table 3.

TABLE 3

| | Ingredient | Amount (mg) |
|---|---|---|
| Acetyl L- | Acetyl L-carnitine HCl | 590 |
| carnitine granule | | (=500 mg acetyl |
| | | L-carnitine) |
| | Microcrystalline cellulose | 35 |
| | Polyvinylpyrrolidone | 56 |
| MTHF granule | Methylfolate calcium salt | 7.5 |
| GR15 | Ascorbic acid | 33 |
| | Microcrystalline cellulose | 190 |
| | Hydroxypropylmethylcellulose | 12 |
| | Magnesium stearate | 10 |
| | Colloidal silica | 3 |
| Film coating | Opadry AMB II | 40 |

In another aspect, the composition in tablet form may comprise vitamins, which may be selected from water-soluble vitamins and fat-soluble vitamins or mixtures thereof.

The water-soluble vitamins are selected from the group comprising vitamin B1 (thiamine or aneurine), vitamin B2 (riboflavin or lactoflavin), vitamin B3 or vitamin PP (niacin or nicotinic acid), vitamin B5 or vitamin W (pantothenic acid), vitamin B6 or vitamin Y (pyridoxine or pyridoxamine or pyridoxal), vitamin B8 or vitamin H or vitamin I (biotin), vitamin B9 or vitamin BC or vitamin M (folic acid or pteroyl(mono)glutamic acid or folacin), and vitamin B12 (cobalamin).

The fat-soluble vitamins are selected from the group comprising vitamin A (retinol and retinoids), vitamin D (D2: ergocalciferol, D3: cholecalciferol), vitamin E (tocopherol), vitamin K (K1: naphthoquinone, K2: phylloquinone, K3: menaquinones, menadione), vitamin F (alpha-linolenic acid, Omega 3) and vitamin Q (ubiquinone, coenzyme Q).

The vitamins may be included in the form of powder and/or granules.

In one aspect, the composition comprises an amount of MTHF granules ranging from 5 to 40% (w/w), acetyl L-carnitine, preferably HCl, granules ranging from 50 to 90% (w/w), water-soluble vitamins ranging from 0 to 10% (w/w), a lubricant ranging from 0.1 to 5% (w/w), a glidant ranging from 0.1 to 1% (w/w), and a diluent ranging from 0 to 10% (w/w), relative to the weight of the finished tablet, and the tablet may optionally be film coated.

The tablet may be coated with a coating designed to achieve controlled release of the active ingredients.

In one aspect, the composition comprises an amount of MTHF granules ranging from 5 to 30% (w/w), acetyl L-carnitine, preferably HCl, granules ranging from 60 to 90% (w/w), water-soluble vitamins ranging from 1 to 5% (w/w), a lubricant ranging from 1 to 5% (w/w), a glidant ranging from 0.1 to 1% (w/w), and a diluent ranging from 0 to 10% (w/w), on the weight of the tablet, and the tablet may optionally be film coated.

In another aspect the tablet composition comprises MTHF granules ranging from 50 to 500 mg, acetyl L-carnitine, preferably HCl, granules ranging from 500 to 800 mg, water-soluble vitamins ranging from 2 to 50 mg, lubricant ranging from 1 to 50 mg, glidant ranging from 1 to 10 mg, and diluent ranging from 1 to 100 mg, together with pharmaceutically acceptable excipients.

In another aspect, the tablet composition comprises 400 to 700 mg of acetyl L-carnitine hydrochloride granules, MTHF granules corresponding to an amount of MTHF ranging from 2 to 25 mg, vitamin B6 ranging from 10 to 50 mg, vitamin B12 ranging from 1 to 10 mg, ascorbic or citric acid ranging from 5 to 50 mg, lubricant ranging from 1 to 50 mg, glidant ranging from 1 to 10 mg, and diluent ranging from 0 to 100 mg.

In a particular aspect, the unit composition in tablet form is shown in Table 4.

TABLE 4

| | Ingredient | Amount (mg) |
|---|---|---|
| Acetyl L- | Acetyl L-carnitine HCl | 590 |
| carnitine granule | | (=500 mg acetyl |
| | | L-carnitine) |
| | Microcrystalline cellulose | 35 |
| | Polyvinylpyrrolidone | 56 |
| MTHF granule | Methylfolate calcium salt | 3 |
| GR3 | Ascorbic acid | 6 |
| | Corn starch FU | 72 |
| | Pregelatinized starch | 5 |
| Extragranular excipient | Vitamin B6 | 35 |
| Extragranular excipient | Vitamin B12 | 2 |
| Extragranular excipient | Corn starch FU | 48 |
| Extragranular excipient | Magnesium stearate | 10 |
| Extragranular excipient | Colloidal silica | 3 |
| Film coating | Opadry AMB II | 35 |

The tablet comprising MTHF granules in an amount from 5 to 40% (w/w) and acetyl L-carnitine, preferably HCl, granules (w/w) in an amount from 50 to 90%, according to the invention has the advantage of being stable when stored at 25° C., RH 60%, for at least 6 months, and at 40° C., RH 75%, for 6 months, stability being defined as maintaining the MTHF assay value higher than 90%.

The tablet of the invention comprising MTHF granules in an amount from 5 to 40% (w/w) and acetyl L-carnitine granules, preferably HCl, in an amount from 50 to 90% (w/w), is characterized by a water content determined by the Karl Fischer method lower than 5%, a hardness value ranging between 4 and 20 Kp and a friability value ranging between 0.1 and 1%.

The compositions comprising MTHF in the form of granules together with pharmaceutically acceptable excipients, and optionally other active ingredients, have the advantage of not being subject to degradation. MTHF in granules remains stable even in the presence of hydrated ingredients which usually lead to its degradation, such as vitamins or salts.

The compositions comprising MTHF in granules give rise to recovery of the assay value compared with TO, unlike the compositions wherein MTHF is present in tablet compositions in the form of methylfolate calcium salt powder and subject to direct compression (comparative examples).

The composition comprising MTHF granules is stable, and the MTHF assay value is maintained for at least six months at a temperature of 25° C., without any methylfolate degradation.

Another aspect of the invention is a process for obtaining a tablet composition comprising MTHF granules in an amount from 5 to 40% (w/w) and acetyl L-carnitine, preferably HCl, granules in an amount from 50 to 90% (w/w), in comparison to the finished composition.

The process for the preparation of the tablet according to the invention comprises:

a) preparing MTHF granules;

b) preparing a carnitine derivative salt, preferably acetyl L-carnitine, preferably HCl, granules;

c) mixing the granules obtained in steps a) and b) with extragranular excipients;

d) compressing the mixture obtained in step c), and

e) optionally film coating.

According to a preferred embodiment, the process comprises:
mixing MTHF granules and carnitine derivative salt, preferably acetyl L-carnitine, preferably HCl, granules with extragranular excipients;
compressing the so obtained mixture, and
optionally film coating.

The granules obtained according to step a) may optionally be used and mixed with other active ingredients and with pharmaceutically acceptable excipients for the preparation of compositions in single-dose sachets.

The MTHF granules may be prepared by dry or wet granulation, mixing MTHF in an amount ranging from 3 to 10% (w/w) with an amount ranging from 3 to 15% (w/w) of antioxidant or stabilizer, an amount ranging from 50 to 90% (w/w) of diluent and an amount ranging from 4 to 10% (w/w) of binder, on the weight of the granulate.

The preparation of carnitine granules includes granulation of a carnitine derivative salt, preferably acetyl L-carnitine, more preferably hydrochloride, ranging from 75 to 90% (w/w) with microcrystalline cellulose ranging from 5 to 10% (w/w) and polyvinylpyrrolidone ranging from 5 to 10% (w/w) on the weight of the granulate.

The resulting granules are preferably sieved through a 400 to 800 μm mesh screen.

The MTHF granules are mixed with the carnitine granules, preferably acetyl L-carnitine hydrochloride granules, in a weight ratio ranging from 1:3 to 1:10, and the resulting homogeneous mixture is mixed with the extragranular excipients.

The MTHF granules, in an amount ranging from 5 to 40% (w/w), are mixed with an amount of carnitine granules, preferably acetyl L-carnitine granules, ranging from 55 to 85% (w/w), and a lubricant in an amount ranging from 0.1 to 5% (w/w), glidants ranging from 0.1 to 5% (w/w) and a diluent ranging from 0 to 10% (w/w), are added to the homogeneous mixture. Other pharmaceutically acceptable excipients, and natural or synthetic active ingredients, may be added to the mixture.

B vitamins, in an amount ranging from 0 to 10% (w/w) of the weight of the finished composition, may be added to the mixture of MTHF granules and carnitine granules, preferably acetyl L-carnitine salt thereof obtained in step c). Finally, the homogeneous mixture is compressed, and the resulting tablets are film coated.

The present invention has demonstrated in a in vivo depression animal model, that the combination of MTHF and LAC (Acetyl L-carnitine) is efficacious in the treatment of depression with a synergistic effect.

The animal study was performed in a validate experimental model subject to Chronic Unpredictable Stress (CUS), as described in J H Cryan et al. in J. Neubiorev. 2005, 03, 009, for a period of 4 weeks to induce the depressive phenotype CUS. The CUS allows the depressive phenotype responsive to standard antidepressant treatments to be reproduced in vivo in mammals. The animals were divided in five groups and the group which received the combination of MTHF with LAC was compared with the groups which received MTHF and LAC separately. All the groups were compared with the group which received saline solution and the group which not submitted to CUS.

The mice were subjected to CUS for 4 weeks, and the drug treatment started at the 3rd week of CUS and maintained until the end of the 4-week treatment.

To assess the antidepressant effects of the selected product LAC and MTHF the animals were submitted to the Forced swimming test (“FST”) as described in J H Cryan et al. in J. Neubiorev. 2005, 03, 009 and measured the Total Immobility Time expressed in the four group of animals.

It resulted that the animal groups after 3 day treatment of MTHF with LAC, the immobility time of forced swimming was significantly reduced in comparison to the other treatment groups.

The animal study confirms the efficacy of MTHF and LAC in a model of depression and the efficacy of said combination may be transferred to a human dosage the of MTHF from 5 to 95 mg/day and LAC at a dosage from 100 to 1000 mg/day, which can be administered one or two time a day, alone or in combination with other antidepressive compounds.

From the obtained results it is possible to state that:

1) the exposure to chronic stress (CUS) produced an increase in the Total Immobility Time when compared to control unstressed conditions, as shown by the comparison between Group I treated with saline solution and Group II treated with saline and submitted to CUS;

2) the effects of chronic stress (CUS) was not antagonised by a 3 mg/kg MTHF, as shown by Group III treated with MTHF in comparison with the control group (Group III);

3) the effects of chronic stress (CUS) was not antagonised by the 30 mg/kg LAC, as shown by the group treated with LAC (Group IV) in comparison to the control group (Group II);

4) the effects of chronic stress (CUS) was antagonised by the co-administration of LAC+MTHF at the same doses, respectively, as shown by the comparison between Group II and Group V.

Interestingly, the mice in Group treated with 3 mg/kg MTHF and LAC 30 mg/kg showed that their Total Immobility Time was not different from those of the control unstressed mice reported control group (Group I).

These data indicate that the co-administration of LAC+MTHF was able to reduce the Total Immobility Time when tested at doses that, when administered alone, did not improve the FTS performance. The in vivo study has demonstrated a significant antidepressant-like effect given by the co-administered of LAC and MTHF.

Mice after 4 weeks of CUS and 2 week of pharmacological treatments, as described before, at the end of Week 5 the animals were sacrificed and the level of BDNF protein were measured in Frontal Cortex by western blot.

BDNF is an important neuronal trophic factor whose reduction in levels within limbic structures (i.e., Frontal Cortex and Hippocampus) has been related to the global attenuation of neuroplasticity produced by chronic stress and depression (Licznerski et Jonas, Proc Natl Acad Sci USA.; 115(15): 3742-3744, 2018).

BDNF levels increased in both limbic structures in Frontal Cortex of mice exposed to CUS after co-treatment with the combination of LAC 30 mg/Kg with MF 3 mg/Kg (Group V) when compared to saline-treated control CUS mice (Group II). These data demonstrate that the co-administration of LAC+MTHF produced an effect on BDNF that is compatible with the behavioural results of antidepressant-like effect produced by the LAC+MTH co-treatments in the same experiment.

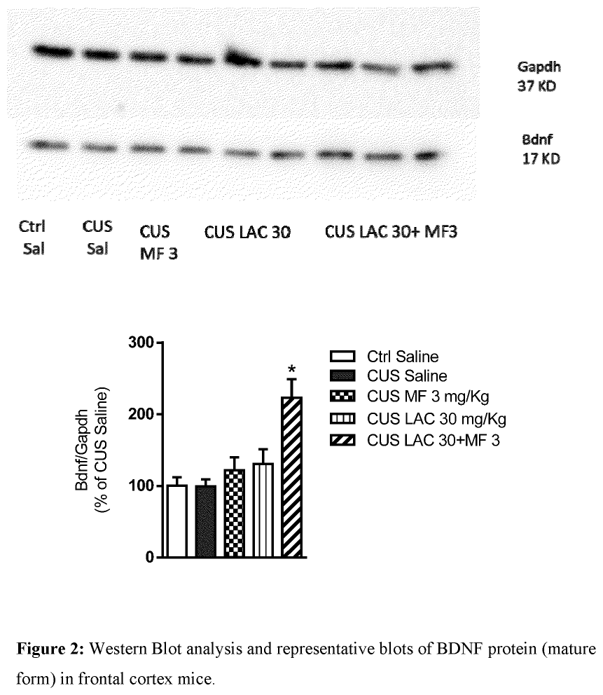

FIG. 2 shows the BDNF protein expression levels measured in the mouse Frontal Cortex by western blot. The BDNF levels in the mouse Frontal Cortex showed an increase in the group treated with 30 mg/kg LAC ang MTHF 3 mg/Kg (Group V), in comparison with the groups treated with LAC and MTHF separately (Groups III and IV).

The composition containing MTHF granules and carnitine granules is useful to affect structural neuronal plasticity process.

Depending on the kind and level of condition, the therapeutic agent may be administered 1 to 6 times per day such as 1, 2, 3, 4, 5 or 6 times a day. If the subject has a risk of having MDD (Major depressive disorder), a combination therapy or adjunctive therapy of a formulation of the present invention and an antidepressant drug can be recommended, selected or administered. In some instances, the antidepressant drug is a selective serotonin reuptake inhibitor (SSRI) or a selective norepinephrine reuptake inhibitor (SNRI). In some cases, the combination therapy or adjunctive therapy includes a folate formulation and a SSRI. Alternatively, the combination therapy or adjunctive therapy includes a folate formulation and a SNRI. Various types or classes of antidepressants are known and commercially available. Non-limiting examples of antidepressant include serotonin reuptake inhibitors (SRIs), serotonin reuptake inhibitors (SSRIs), serotonin and dopamine reuptake inhibitors (SDRIs), serotonin-norepinephrine reuptake inhibitors (SNRIs), serotonin-noradrenaline-dopamine reuptake inhibitors (SNDRIs), noradrenergic and specific serotonergic anti-depressants (NASSAs), norepinephrine-dopamine reuptake inhibitors (NDRIs), norepinephrine (noradrenaline) reuptake inhibitors (NRIs), monoamine oxidase inhibitors (MAOIs), selective serotonin reuptake enhancers (SSREs), melatonergic agonists, tryptamines, tricyclic antidepressants (TCAs), and atypical antidepressants. SSRIs act to preventing the reuptake of serotonin by the presynaptic neuron, thereby maintaining high levels of serotonin in the synapse

The composition according to the invention is useful for all individuals in whom the administration of MTHF is useful. The composition containing MTHF and acetyl L-carnitine, preferably hydrochloride is useful for treatment and/or prevention in individuals with disorders connected with depression, cognitive or psychotic disorders or in patients suffering from endothelial dysfunctions as neuropathies, peripheral neuropathies and diabetic neuropathies peripheral diabetic neuropathy, as it guarantees the dose necessary to achieve the beneficial effect and represents a useful support for the classic pharmacological treatments indicated in the guidelines.

The composition according to the invention may be administered for use in depression diseases at a dosage of 1, 2 or 3 tablets 1, 2, 3 or 4 times a day, with an MTHF dose ranging from 5 to 90 mg/day, and an acetyl L-carnitine dose of 100 mg to 3 grams/day, without any side effects.

The composition according to the invention may be administered as concomitant treatment with antidepressive treatments in use.

The solid composition comprising MTHF granules in an amount ranging from 5 to 40% (w/w) and acetyl L-carnitine hydrochloride granules in an amount ranging from 50 to 90% (w/w), on the weight of the final composition, together with pharmaceutically acceptable excipients, is useful for the treatment and/or prevention of disorders connected with depression or schizophrenia. The composition containing 5 to 20 mg of MTHF granules and 400 to 750 mg of acetyl L-carnitine granules may be administered at a dosage of 1, 2 or 3 tablets 1, 2, 3 or 4 times a day, with a MTHF daily dosage ranging from 5 to 90 mg/day and an acetyl L-carnitine daily dosage ranging from 100 mg to 1000 mg/day for use in disorders connected with depression or schizophrenia.

In particular, the composition according to the invention comprising 7.5 and 15 mg of MTHF granules and 500 mg of acetyl L-carnitine (corresponding to about 590 mg of acetyl L-carnitine hydrochloride) granules is useful for the treatment and prevention of disorders connected with depressive states.

Also Vitamin of B group may be comprised in the composition containing MTHF and LAC, wherein Vitamins of the B group in an amount from 0.1 to 10% (w/w) are contained in the composition with MTHF granules in an amount ranging from 5 to 40% (w/w) and acetyl L-carnitine hydrochloride granules in an amount ranging from 50 to 90% (w/w) in comparison to the final composition, said composition, is useful for the treatment and/or prevention of disorders related to neuropathies, in particular peripheral neuropathies or diabetic neuropathies.

The composition containing 2 to 6 mg of MTHF granules, 100 to 1000 mg of acetyl L-carnitine granules, and vitamins, in particular B vitamins such as B6 and/or B12, in an amount ranging from 2 to 50 mg, may be administered at the rate of 1, 2 or 3 tablets 1, 2, 3 or 4 times a day, giving a dose of MTHF ranging from 2 to 24 mg/day, a dose of acetyl L-carnitine ranging from 400 mg to 3 g/day, and vitamins B6 and B12 ranging from 2 to 200 mg/day, for use in the treatment or prevention of neuropathy.

The composition according to the invention comprising 3 mg of MTHF granules, 500 mg of acetyl L-carnitine granules, and vitamins, in particular B vitamins such as B6 and/or B12, in an amount ranging from 2 to 50 mg, is useful for the treatment and/or prevention of neuropathies such as peripheral neuropathies or diabetic neuropathies. The tablets may be administered 1, 2, 3 or 4 times a day, giving a daily dose of 2 to 12 mg of MTHF, 0.5 to 2 g of acetyl L-carnitine and 2 to 140 mg of B vitamins.

The composition is effective, well tolerated and has no side effects.

A synergistic effect may be obtained thanks to the concomitant use of the compositions comprising methylfolate in the form of granules according to the invention with the medicinal products in use for the treatment of depression.

The following examples further illustrates the invention.

### EXAMPLES

### Example 1: Preparation of Acetyl L-Carnitine Hydrochloride Granules

590 g of acetyl L-carnitine hydrochloride was wet granulated with 35 g of microcrystalline cellulose and 56 g of polyvinylpyrrolidone using a fluid bed. The granules were dried and sieved through a 600 μm mesh screen.

### Example 2: Preparation of Calcium L-5-Methylfolate (MTHF) Granules

The calcium methylfolate granules were obtained by dry granulation and wet granulation processes.

a) Dry Granulation

The granule preparation ingredients were placed in a mixer in the amounts shown in Table 5 and then mixed. The resulting mixtures were granulated in a dry compactor. The granules were ground and sieved through 600 μm mesh screens, and the resulting granules were used.

TABLE 5

| Ingredient | GR1 (g) | GR2 (g) |
|---|---|---|
| Calcium L-5- methylfolate | 30 | 30 |
| Ascorbic acid | — | 260 |
| Mannitol | 710 | 710 |
| Magnesium stearate | 5 | 5 |

The GR1 and GR2 granules have a bulk density (BD) of 0.68 g/ml and a tap density (TD) of 0.79 g/ml.

The GR1 and GR2 granules are characterized by a particle size distribution (PSD) wherein:

10%<125 μm; 50%<425 μm; 90%<600 μm.

b) Wet Granulation

Calcium L-5-methylfolate was placed in a high-shear mixer together with the proportional amounts of the various ingredients, in the ratios shown in Table 6.

TABLE 6

| | GR3 | GR4 | GR5 | GR6 | GR15 |
|---|---|---|---|---|---|
| Ingredient | (g) | (g) | (g) | (g) | (g) |
| L-5- methylfolate | 30 | 15 | 15 | 15 | 7.5 |
| calcium salt | | | | | |
| Ascorbic acid | 30 | 16.5 | — | 16.5 | 16.5 |
| Citric acid | — | — | 16.5 | — | — |
| Corn starch FU | 720 | 172 | 172 | — | — |
| Microcrystalline | — | — | — | 183 | 190 |
| cellulose | | | | | |
| PH101 | | | | | |
| Pregelatinized starch | 25 | 11.5 | 11.5 | — | — |
| (Starch ® 1500) | | | | | |
| Hydroxypropylcellulose | — | — | — | 6 | 6 |

At the same time, an aqueous solution containing the stabilizer/antioxidant (ascorbic acid or citric acid) and the binder (modified starch or hydroxypropylcellulose) was prepared in a homogenizer in the amounts shown in Table 5, and the binder solution was added to the solid mixture.

The resulting granules were placed to dry in a fluid-bed apparatus.

The resulting granules were then ground and sieved through a 600 μm mesh screen.

The GR3, GR4 and GR5 granules have a bulk density (BD) of 0.58 g/ml and a tap density (TD) of 0.67 g/ml.

The GR3, GR4 and GR5 granules are characterized by a particle size distribution (PSD) wherein: 10%<53 μm; 50%<180 μm; 90%<425 μm.

The GR6 granule has a bulk density (BD) of 0.38 g/ml and a tap density (TD) of 0.46 g/ml.

The GR6 granule is characterized by a particle size distribution (PSD) wherein: 10%<53 μm; 50%<180 μm; 90%<425 μm.

The granules obtained may be used immediately or stored for use in solid preparations.

### Example 3: Determination of Stability of Calcium Methylfolate Granules

The stability of GR1-GR6 granules prepared in Example 2 was tested at 40±2° C., RH 75%, for one month.

The MTHF assay value in the GR1-GR6 granules was determined by HPLC using a standard curve. A Spherisorb-SCX 4.6×250 mm chromatography column was used, with 5 μm particles; the MTHF was eluted under isocratic conditions with 50 mM KH₂PO₄/CH₃CN (32/68) eluent at a pH of 2.5; flow rate 1.2 mL/min and 220 nm wavelength UV detector.

The assay value is expressed as percentage recovery of methylfolate compared with T0.

Table 7 shows the assay value of the GR1-GR6 granules at 40° C., RH 75%.

TABLE 7

| | MTHF assay value |
|---|---|
| | T = 1 month |
| GR1 | 95.4% ± 3.2 |
| GR2 | 97.4% ± 2.5 |
| GR3 | 91.7% ± 3.1 |
| GR4 | 101.6% ± 3.9 |
| GR5 | 91.5% ± 4.1 |
| GR6 | 95.4% ± 4.6 |

### Example 4: Preparation of Granules Comprising Methylfolate and Vitamins B6 and B12

a) Dry Granulation

The granule preparation ingredients were placed in a mixer in the amounts shown in Table 8. The resulting mixtures were granulated in a dry compactor. The granules were ground and sieved through 600 μm mesh screens, and used.

TABLE 8

| Ingredient | GR7 (g) |
|---|---|
| L-5 methylfolate calcium salt | 3 |
| Vitamin B6 | 35 |
| Vitamin B12 | 2 |
| Mannitol | 100 |
| Magnesium stearate | 0.8 |

b) Wet Granulation

The granules reported in Table 9 were prepared.

TABLE 9

| Ingredient | GR8(g) | GR9(g) | GR10(g) | GR11(g) | GR12(g) | GR13(g) |
|---|---|---|---|---|---|---|
| Acetyl L-carnitine | | | | | | 590 |
| HCl | | | | | | |
| L-5 methylfolate | 3 | 3 | 3 | 3 | 3 | 3 |
| calcium salt | | | | | | |
| Vitamin B6 | 35 | 35 | | | 35 | 35 |
| Vitamin B12 | 2 | 2 | 2 | 2 | 2 | 2 |
| Ascorbic acid | | 30 | 6 | | | 15 |
| Citric acid | | | | | 24 | |
| Acetyl cysteine | | | | 6 | | |
| Mannitol | 250 | 250 | | | | |
| Corn starch FU | | | 50 | 50 | | 60 |
| Modified starch | | | | | 60 | 8 |
| PVP | 28 | 28 | | | | |

Preparation of GR8, GR9, GR10 and GR11: L-5-methylfolate calcium salt, vitamin B6, vitamin B12 and diluent (mannitol or corn starch FU) were placed in a high-shear mixer in the amounts shown in Table 9. The mixture was granulated with the granulating solution prepared by solubilizing the binder or stabilizer/antioxidant (PVP or ascorbic acid or acetylcysteine, in the amounts shown in Table 9) in demineralized water, then dried in an oven or fluid-bed dryer until reaching a water content <5%. The resulting granulate was ground through a 600 μm mesh screen.

Preparation of GR12: L-5-methylfolate calcium salt was solubilized in water in the presence of citric acid. Modified starch and vitamins B6 and B12 were added to the solution, in the amounts shown in Table 9. The resulting mixture was freeze-dried and pulverized through a 600 μm mesh screen.

Preparation of GR13: L-5-methylfolate calcium salt, acetyl L-carnitine hydrochloride, vitamin B6, vitamin B12 and corn starch FU were placed in a high-shear mixer in the amounts shown in Table 9. At the same time, a granulating solution was prepared by dispersing modified starch and ascorbic acid in demineralized water using an UltraTurrax or Silverson homogenizer. The mixture was granulated, and dried until a water content of <5% was reached. The granules were ground and dried on 600 μm mesh sieves.

The stability of GR8-GR13 granules was tested at 40±2° C., RH 75%, for 1 month.

The MTHF assay value in the GR8-GR13 granules was determined by HPLC. A Spherisorb-SCX 4.6×250 mm, 5 μm chromatography column was used; the MTHF was eluted under isocratic conditions with eluent 50 mM KH₂PO₄/CH₃CN (32/68) at pH=2.5, flow rate=1.2 mL/min with a 220 nm wavelength UV detector.

The assay value is expressed as percentage recovery compared with TO.

The stability of MTHF in the GR8-GR13 granules at 40° C., RH 75%, is shown in Table 10.

TABLE 10

| | MTHF assay value |
|---|---|
| | T = 1 month |
| GR8 | 62.8% ± 4.1 |
| GR9 | 89.0% ± 4.3 |
| GR10 | 99.9% ± 3.1 |
| GR11 | 101.1% ± 3.9 |
| GR12 | 66.5% ± 2.8 |
| GR13 | 70.3% ± 3.3 |

### Example 5: Preparation of Granules Comprising Vitamins B6 and B12

The granule preparation ingredients were placed in a mixer in the amounts shown in Table 10, and then mixed. The resulting mixtures were granulated in a dry compactor. The granules were ground and sieved through 600 μm mesh screens, and the resulting granules were used.

The composition of the granules is shown in Table 11.

TABLE 1

| Ingredient | GR14 (g) |
|---|---|
| Vitamin B6 | 350 |
| Vitamin B12 | 20 |
| Mannitol | 710 |
| Magnesium stearate | 5 |

### Example 6: Preparation of Tablets Containing Methylfolate Calcium Salt and Acetyl L-Carnitine (Tablet 1)

681 g of acetyl L-carnitine hydrochloride granule according to Example 1 was placed in a mixer, and an amount corresponding to 243 g of calcium methylfolate granule according to preparation GR4 was added. Ten grams of magnesium stearate and 3 g of colloidal silica were added to the homogeneous mixture. The mixture was stirred for 5 minutes (20 rpm) and then compressed in a tablet press. The resulting tablets were coated with Opadry AMB II.

The tablets have the composition per unit shown in Table 12.

TABLE 12

| | Ingredient | Amount (mg) | % (w/w) |
|---|---|---|---|
| Acetyl L- | Acetyl L-carnitine HCl | 590 | 60.4 |
| carnitine | | (=500 mg | |
| granule | | acetyl | |
| | | L-carnitine) | |
| | Microcrystalline cellulose | 35 | 3.6 |
| | Polyvinylpyrrolidone | 56 | 5.7 |
| Methylfolate | Calcium methylfolate | 15 | 1.5 |
| granule GR4 | Ascorbic acid | 33 | 3.4 |
| | Starch | 172 | 17.6 |
| | Modified starch | 23 | 2.3 |
| | Magnesium stearate | 10 | 1.0 |
| | Colloidal silica | 3 | 0.31 |
| Film coating | Opadry AMB II | 40 | 4.1 |

The uncoated tablets are characterized by a water content of 1.8% determined by the Karl Fischer method, a hardness value of 10±3 Kp and a friability value of 0.4%.

The stability of coated tablets (Tablet 1) was tested for 6 months at 25±2° C., RH 60%, and 40±2° C., RH 75%, and the methylfolate content was determined by comparison with TO as reported in Example 10.

### Example 7: Preparation of Tablets Containing Methylfolate Calcium Salt and Acetyl L-Carnitine Hydrochloride (Tablet 2)

681 g of acetyl L-carnitine hydrochloride granules according to Example 1 was placed in a mixer, and an amount corresponding to 243 g of calcium methylfolate granules obtained according to preparation GR5 was added. 10 g of magnesium stearate and 3 g of colloidal silica were added to the homogeneous mixture. The mixture was stirred for 5 minutes (20 rpm) and then compressed in a tablet press. The resulting tablets were coated with Opadry AMB II.

The tablets have the composition per unit shown in Table 13.

TABLE 13

| | Ingredient | Amount (mg) | % (w/w) |
|---|---|---|---|
| Acetyl L- | Acetyl L-carnitine HCl | 590 | 60.4 |
| carnitine | | (=500 mg | |
| granule | | acetyl | |
| | | L-carnitine) | |
| | Microcrystalline cellulose | 35 | 3.6 |
| | Polyvinylpyrrolidone | 56 | 5.7 |
| MTHF granule | Methylfolate calcium salt | 15 | 1.5 |
| GR5 | Citric acid | 33 | 3.4 |
| | Corn starch | 172 | 17.6 |
| | Pregelatinized starch | 23 | 2.3 |
| | Magnesium stearate | 10 | 1.0 |
| | Colloidal silica | 3 | 0.3 |
| Film coating | Opadry AMB II | 40 | 4.1 |

The uncoated tablets are characterized by a water content of 1.8%, determined by the Karl Fischer method, a hardness value of 17±3 Kp and a friability value of 0.2%.

The stability of coated tablets (Tablet 2) was tested for 6 months at 25±2° C., RH 60%, and 40±2° C., RH 75%, and the methylfolate content was determined compared with TO as reported in Example 10.

### Example 8: Preparation of Tablets Containing Methylfolate Calcium Salt and Acetyl L-Carnitine Hydrochloride (Tablet 3 and Tablet 12)

681 g of acetyl L-carnitine hydrochloride granule according to Example 1 was placed in a biconical mixer, and an amount corresponding to 243 g of calcium methylfolate granule obtained according to preparation GR6 was added. 10 g of magnesium stearate and 3 g of colloidal silica were added to the homogeneous mixture. The mixture was stirred for 5 minutes (20 rpm) and then compressed in a tablet press. The resulting tablets were coated with Opadry AMB II.

The tablets have the composition per unit shown in Table 14.

TABLE 14

| | | Tablet | Tablet | |
|---|---|---|---|---|
| | | 3 | 12 | |
| | | Amount | Amount | % |
| | Ingredient | (mg) | (mg) | (w/w) |
| Acetyl L- | Acetyl L-carnitine HCl | 590 | 590 | 60.4 |
| carnitine | | (=500 | (=500 | |
| granule | | mg | mg | |
| | | acetyl L- | acetyl L- | |
| | | carnitine) | carnitine) | |
| | Microcrystalline cellulose | 35 | 35 | 3.6 |
| | Polyvinylpyrrolidone | 56 | 56 | 5.7 |
| MTHF | Methylfolate calcium salt | 15 | — | 1.5 |
| granule | Ascorbic acid | 33 | — | 3.4 |
| GR6 | Microcrystalline cellulose | 183 | — | 18.7 |
| | Hydroxypropyl- | 12 | — | 1.2 |
| | methylcellulose | | | |
| MTHF | Methylfolate calcium salt | — | 7.5 | 0.77 |
| granule | Ascorbic acid | — | 33 | 3.4 |
| GR15 | Microcrystalline cellulose | — | 190 | 19.5 |
| | Hydroxypropyl- | — | 12 | 1.2 |
| | methylcellulose | | | |
| | Magnesium stearate | 10 | 10 | 1.0 |
| | Colloidal silica | 3 | 3 | 0.3 |
| Film | Opadry AMB II | 40 | 40 | 4.1 |
| coating | | | | |

The uncoated tablets are characterized by a water content of 1.8% determined by the Karl Fischer method, a hardness value of 12±1 Kp and a friability value of 0.3%.

The stability of coated tablets (Tablet 3) was tested for 6 months at 25±2° C., RH 60%, and 40±2° C., RH 75%, and the methylfolate assay value was determined over time compared with TO as reported in Example 10.

### Example 9: Preparation of Tablets Containing Methylfolate Calcium Salt and Acetyl L-Carnitine Hydrochloride (Tablet 4—Comparative Example)

590 g of acetyl L-carnitine hydrochloride was placed in a mixer and 15 g of calcium methylfolate was added, together with 35 g of cellulose microcrystalline, 56 g of polyvinylpyrrolidone, 33 g of ascorbic acid, 172 g of corn starch and 23 g of modified starch. Ten grams of magnesium stearate and 3 g of colloidal silica were added to the homogeneous mixture. The mixture was stirred for 5 minutes (20 rpm) and then compressed in a tablet press. The resulting tablets were coated with Opadry AMB II.

The tablets have the composition per unit shown in Table 15.

TABLE 15

| Ingredient | Amount (mg) | % (w/w) |
|---|---|---|
| Acetyl L-carnitine | 590 | 60.4 |
| | (=500 mg acetyl L-carnitine) | |
| Microcrystalline cellulose | 35 | 3.6 |
| Polyvinylpyrrolidone | 56 | 5.7 |
| Calcium methylfolate | 15 | 1.5 |
| Ascorbic acid | 33 | 3.4 |
| Starch | 172 | 17.6 |
| Modified starch | 23 | 2.3 |
| Magnesium stearate | 10 | 1.0 |
| Colloidal silica | 3 | 0.3 |
| Opadry AMB II | 40 | 4.1 |

The stability of coated tablets (Tablet 4) was tested for 2 months at 25±2° C., RH 60%, and 40±2° C., RH 75%, and the methylfolate content was determined over time compared with TO, as reported in Example 10.

### Example 10: Stability of Compositions in Tablets 1-4

Stability of tablets 1-4 was tested at 25±2° C., RH 60% for 12 months, and at 40±2° C., RH 75%, for 6 months.

The methylfolate assay value was determined by HPLC using a Spherisorb SCX 4.6×250 mm column, with a particle size of 5 μm, and a 220 nm wavelength UV detector. Methylfolate was eluted under isocratic conditions with eluent 50 mM KH₂PO₄/CH₃CN: (32/68) at pH 2.5.

The stability is expressed as percentage recovery compared with TO and reported in Tables 16 and 17.

TABLE 16
Methylfolate assay value, T = 25 ± 2° C., RH 60%

| | T1 | T2 | T3 | T4 | T5 | |
|---|---|---|---|---|---|---|
| | (2 | (3 | (6 | (9 | (12 | |
| | months) | months) | months) | months) | months) | SD |
| Tablet 1 | 96.6% | 96.7% | 95.2% | | | 4.8 |
| Tablet 2 | 95.0% | 99.5% | 98.5% | | | 5.4 |
| Tablet 3 | 90.6% | 94.7% | | 94.40% | 91.94% | 5.3 |
| Tablet 4 | 90.00% | 87.00% | 85.00% | | | 2.1 |
| Comparative | | | | | | |

TABLE 17
Methylfolate assay value, T = 40 ± 2° C., RH 75%

| | T1 | T1 | T2 | T3 | |
|---|---|---|---|---|---|
| | (1 month) | (2 months) | (3 months) | (6 months) | SD |
| Tablet 1 | 99.8% | 98.5% | 97.8% | | 4.8 |
| Tablet 2 | 100.1% | 98.0% | 99.3% | | 5.4 |
| Tablet 3 | 100.1% | 88.0% | 96.1% | 88.88% | 5.3 |
| Tablet 4 | 80.00% | 75.00% | 65.00% | | 2.1 |
| Comparative | | | | | |
| example | | | | | |

### Example 11: Preparation of Tablets Containing Methylfolate Calcium Salt, Acetyl L-Carnitine and Vitamins (Tablet 5—Comparative Example)

681 g of acetyl L-carnitine granules prepared according to Example 1, 3 g of calcium methylfolate, 35 g of vitamin B6 and 2 g of vitamin B12 were mixed using the geometric dilution technique for 45 minutes. Fourteen grams of magnesium stearate and 3 grams of colloidal silica were added to the homogeneous mixture. The mixture was stirred for 5 minutes (20 rpm) and then compressed in a tablet press. The resulting tablets were coated with Opadry AMB II.

The composition per unit of Tablet 5 is shown in Table 18.

TABLE 18

| Ingredient | Amount (mg) | % (w/w) |
|---|---|---|
| Acetyl L-carnitine granule | 681 (=500 mg acetyl L- | 65 |
| | carnitine) | |
| Calcium methylfolate | 3 | 0.3 |
| Vitamin B6 | 35 | 4.5 |
| Vitamin B12 | 2 | 0.2 |
| Magnesium stearate | 14 | 1.8 |
| Colloidal silica | 3 | 0.3 |
| Opadry AMB II | 32 | 4.1 |

The uncoated tablets are characterized by a water content of 1.7% determined by the Karl Fischer method, a hardness value of 12±2 Kp and a friability value of 0.7%.

The stability of coated tablets (Tablet 5) was tested for 6 months at 25±2° C., RH 60%, and 40±2° C., RH 75%, and the methylfolate content was determined over time, compared with TO as reported in Example 18.

### Example 12: Preparation of Tablets Containing Methylfolate Calcium Salt, Acetyl L-Carnitine and Vitamins (Tablet 6—Comparative Example)

681 g of acetyl L-carnitine granules prepared according to Example 1, 3 g of calcium methylfolate, 35 g of vitamin B6, 2 g of vitamin B12 and 50 g of ascorbic acid were mixed for about 45 minutes. Fourteen grams of magnesium stearate and 3 grams of colloidal silica were added to the homogeneous mixture. The mixture was stirred for 5 minutes (20 rpm) and then compressed in a tablet press. The resulting tablets were coated with Opadry AMB II.

The composition per unit of Tablet 6 is shown in Table 19.

TABLE 19

| Ingredient | Amount (mg) | % (w/w) |
|---|---|---|
| Acetyl L-carnitine granule | 681 (=500 mg acetyl L- | 61 |
| | carnitine) | |
| Calcium methylfolate | 3 | 0.3 |
| Ascorbic acid | 50 | 6.0 |
| Vitamin B6 | 35 | 4.3 |
| Vitamin B12 | 2 | 0.2 |
| Magnesium stearate | 14 | 1.7 |
| Colloidal silica | 3 | 0.3 |
| Opadry AMB II | 32 | 4.1 |
| Total | 820 | |

The uncoated tablets are characterized by a water content of 1.2% determined by the Karl Fischer method, a hardness value of 10±2 Kp and a friability value of 0.9%.

The stability of coated tablets (Tablet 6) was tested for 6 months at 25±2° C., RH 60%, and 40±2° C., RH 75%, and the methylfolate content was determined over time compared with TO as reported in Example 18.

### Example 13: Preparation of Tablets Containing Methylfolate Calcium Salt, Acetyl L-Carnitine and Vitamins (Tablet 7)

681 g of acetyl L-carnitine hydrochloride granules according to Example 1 was placed in a biconical mixer, and amounts corresponding to 74.5 g of calcium methylfolate granule obtained according to preparation GR1, and 108.5 g of granule comprising vitamins B6 and B12 obtained according to preparation GR14, were added. 7 g of magnesium stearate and 3 g of colloidal silica were added to the homogeneous mixture. The mixture was stirred for 5 minutes (20 rpm) and then compressed in a tablet press. The resulting tablets were coated with Opadry AMB II.

The unit composition is shown in Table 20.

TABLE 20

| | | | % |
|---|---|---|---|
| | Ingredient | Amount (mg) | (w/w) |
| Acetyl L- | Acetyl L-carnitine - HCl | 590 | 65 |
| carnitine granule | | (=500 mg | |
| | | acetyl | |
| | | L-carnitine) | |
| | Microcrystalline | 35 | 3.8 |
| | cellulose | | |
| | Polyvinylpyrrolidone | 56 | 6.1 |
| Methylfolate | Calcium methylfolate | 3 | 0.3 |
| granule GR1 | Mannitol | 71 | 7.8 |
| | Magnesium stearate | 0.5 | 0.1 |
| Vitamin granule | Vitamin B6 | 35 | 3.8 |
| GR14 | Mannitol | 71 | 7.8 |
| | Vitamin B12 | 2 | 0.2 |
| | Magnesium stearate | 0.5 | 0.1 |
| | Colloidal silica | 3 | 0.3 |
| | Magnesium stearate | 7 | 0.8 |
| Film coating | Opadry AMB II | 37 | 4.0 |

The uncoated tablets are characterized by a water content of 1.5% determined by the Karl Fischer method, a hardness value of 19±3 Kp and a friability value of 0.1%.

The stability of coated tablets (Tablet 7) was tested for 6 months at 25±2° C., RH 60%, and at 40±2° C., RH 75%, and the methylfolate content was determined over time compared with TO as reported in Example 18.

### Example 14: Preparation of Tablets Containing Methylfolate Calcium Salt, Acetyl L-Carnitine and Vitamins (Tablet 8)

681 g of granules of acetyl L-carnitine hydrochloride according to Example 1, an amount corresponding to 100.5 g of calcium methylfolate granule obtained according to preparation GR2, and 108.5 g of granule comprising vitamins B6 and B12 obtained according to preparation GR14, were placed in a mixer. 7 g of magnesium stearate and 3 g of colloidal silica were added to the homogeneous mixture. The mixture was stirred for 5 minutes (20 rpm) and then compressed in a tablet press. The resulting tablets were coated with Opadry AMB II.

The composition per unit of Tablet 8 is shown in Table 21.

TABLE 21

| | Ingredient | Amount (mg) | % (w/w) |
|---|---|---|---|
| Acetyl L-carnitine | Acetyl L-carnitine - | 590 | 63 |
| granule | HCl | (=500 mg | |
| | | acetyl | |
| | | L-carnitine) | |
| | Microcrystalline | 35 | 3.7 |
| | cellulose | | |
| | Polyvinylpyrrolidone | 56 | 6 |
| Methylfolate | Calcium methylfolate | 3 | 0.3 |
| granule | Mannitol | 71 | 7.6 |
| GR2 | Ascorbic acid | 26 | 2.8 |
| | Magnesium stearate | 0.5 | 0.15 |
| Vitamin | Vitamin B6 | 35 | 3.7 |
| granule GR14 | Vitamin B12 | 2 | 0.2 |
| | Mannitol | 71 | 7.6 |
| | Magnesium stearate | 0.5 | 0.1 |
| | Magnesium stearate | 7 | 0.7 |
| | Colloidal silica | 3 | 0.3 |
| Film coating | Opadry AMB II | 37 | 4.0 |
| Total | | 937 | |

The uncoated tablets are characterized by a water content of 1.8% determined by the Karl Fischer method, a hardness value of 19±3 Kp and a friability value of 0.2%.

The stability of coated tablets (Tablet 8) was tested for 6 months at 25±2° C., RH 60%, and 40±2° C., RH 75%, and the methylfolate assay value was determined compared with TO as reported in Example 18.

### Example 15: Preparation of Tablets Containing Methylfolate Calcium Salt, Acetyl L-Carnitine and Vitamins (Tablet 9)

681 g of acetyl L-carnitine hydrochloride granules according to Example 1 was placed in a mixer, and an amount corresponding to 86 g of calcium methylfolate granules obtained according to preparation GR3, 35 g of vitamin B6, and 2 g of vitamin B12 previously diluted in 48 g of corn starch, were added. Ten grams of magnesium stearate and 3 g of colloidal silica were added to the homogeneous mixture. The mixture was stirred for 5 minutes (20 rpm) and then compressed in a tablet press. The resulting tablets were coated with Opadry AMB II.

The composition per unit of Tablet 9 is shown in Table 22.

TABLE 22

| | Ingredient | Amount (mg) | % (w/w) |
|---|---|---|---|
| Acetyl L-carnitine | Acetyl L-carnitine HCl | 590 | 65.6 |
| granule | | (=500 mg | |
| | | acetyl | |
| | | L-carnitine) | |
| | Microcrystalline | 35 | 3.9 |
| | cellulose | | |
| | Polyvinylpyrrolidone | 56 | 6.2 |
| MTHF granule GR3 | Methylfolate calcium | 3 | 0.3 |
| | salt | | |
| | Ascorbic acid | 6 | 0.7 |
| | Corn starch | 72 | 8.0 |
| | Pregelatinized starch | 5 | 0.5 |
| | Vitamin B6 | 35 | 3.9 |
| | Vitamin B12 | 2 | 0.2 |
| | Corn starch FU | 48 | 5.3 |
| | Magnesium stearate | 10 | 1.1 |
| | Colloidal silica | 3 | 0.3 |
| Film coating | Opadry AMB II | 35 | 3.9 |

The uncoated tablets are characterized by a water content of 1.7% determined by the Karl Fischer method, a hardness value of 9±2 Kp and a friability value of 0.4%.

The stability of coated tablets (Tablet 9) was tested for 6 months at 25±2° C., RH 60%, and 40±2° C., RH 75%, and the methylfolate content was determined over time compared with TO as reported in Example 18.

### Example 16: Preparation of Tablets Comprising Methylfolate Calcium Salt, Acetyl L-Carnitine and Vitamins (Tablet 10)

713 g of acetyl L-carnitine hydrochloride granules, calcium methylfolate and vitamins B6 and B12 obtained according to preparation GR13, 8 g of magnesium stearate and 3 g of colloidal silica were placed in a biconical mixer. The mixture was stirred for 5 minutes (20 rpm) and then compressed in a tablet press. The resulting tablets were coated with Opadry AMB II.

The composition per unit of Tablet 5 is shown in Table 23.

TABLE 23

| | | Amount | % |
|---|---|---|---|
| | Ingredient | (mg) | (w/w) |
| Granule GR 13 | Acetyl L-carnitine HCl | 590 | 78.3 |
| | | (=500 mg | |
| | | acetyl | |
| | | L-carnitine) | |
| | Methylfolate calcium salt | 3 | 0.4 |
| | Ascorbic acid | 15 | 2.0 |
| | Corn starch FU | 60 | 8.0 |
| | Modified starch | 8 | 1.0 |
| | Vitamin B6 | 35 | 4.6 |
| | Vitamin B12 | 2 | 0.3 |
| | Magnesium stearate | 8 | 1.0 |
| | Colloidal silica | 3 | 0.4 |
| Film coating | Opadry AMB II | 29 | 3.8 |

The uncoated tablets are characterized by a water content of 1.5% determined by the Karl Fischer method, a hardness value of 5±1 Kp and a friability value of 0.1%.

The stability of coated tablets (Tablet 10) was tested for 6 months at 25±2° C., RH 60%, and 40±2° C., RH 75%, and the methylfolate content was determined over time compared with TO as reported in Example 18.

### Example 17: Preparation of Tablets Comprising Methylfolate, Acetyl L-Carnitine Hydrochloride and Vitamins (Tablet 11)

681 g of acetyl L-carnitine hydrochloride granules according to Example 1 was placed in a mixer, and an amount equal to 140.8 g of calcium methylfolate granules and vitamins B6 and B12 obtained according to preparation GR7 was added. 7 g of magnesium stearate and 3 g of colloidal silica were added to the homogeneous mixture. The mixture was stirred for 5 minutes (20 rpm) and then compressed in a tablet press. The resulting tablets were coated with Opadry AMB II.

The composition per unit of Tablet 5 is shown in 24.

TABLE 24

| | Ingredient | Amount (mg) | % (w/w) |
|---|---|---|---|
| Acetyl L- | Acetyl L-carnitine HCl | 590 | 68.2 |
| carnitine | | (=500 mg | |
| granule | | acetyl | |
| | | L-carnitine) | |
| | Microcrystalline cellulose | 35 | 4.1 |
| | Polyvinylpyrrolidone | 56 | 6.5 |
| MTHF granule | Methylfolate calcium salt | 3 | 0.3 |
| GR7 | Vitamin B6 | 35 | 4.1 |
| | Vitamin B12 | 2 | 0.2 |
| | Mannitol | 100 | 11.6 |
| | Magnesium stearate | 0.8 | 0.1 |
| | Magnesium stearate | 7 | 0.8 |
| | Colloidal silica | 3 | 0.3 |
| Film coating | Opadry AMB II | 33.2 | 3.8 |

The uncoated tablets are characterized by a water content of 1.7% determined by the Karl Fischer method, a hardness value of 19±3 Kp and a friability value of 0.3%.

The stability of coated tablets (Tablet 11) was tested content was determined over time compared with TO as reported in Example 18.

### Example 18: Stability of Tablets 5-11

Stability Tablets 5-11 were tested at 25±2° C., RH 60% for 12 months, and 40±2° C., RH 75%, for 6 months.

The methylfolate assay value was determined by HPLC using a Spherisorb SCX 4.6×250 mm column with a 5 μm particle size, and a 220 nm wavelength UV detector. Methylfolate was eluted under isocratic conditions with eluent 50 mM KH₂PO₄/CH₃CN: (32/68) at pH 2.5.

The stability is expressed as percentage recovery compared with the concentration at TO and reported in Tables 25 and 26.

TABLE 25
Calcium L-5-methylfolate assay value at 25° C., RH 60%

| | Titre % | Titre % | Titre % | Titre % | Titre % | |
|---|---|---|---|---|---|---|
| | T = 2 | T = 3 | T = 6 | T = 9 | T = 12 | |
| | months | months | months | months | months | SD |
| Tablet 5 | 93.2% | 85.1% | 72.8% | | | 2.3 |
| Comparative | | | | | | |
| example | | | | | | |
| Tablet 6 | 94.5% | 95.1% | 93.3% | | | 2.1 |
| Comparative | | | | | | |
| example | | | | | | |
| Tablet 7 | 99.5% | 95.5% | 91.1% | | | 4.8 |
| Tablet 8 | 104.3% | 102.6% | 99.5% | | | 3.2 |
| Tablet 9 | 100.1% | 102.8% | 102.8% | 91.66% | 97.22% | 4.8 |
| Tablet 10 | 89.0% | 80.9% | 67.0% | | | 3.3 |
| Tablet 11 | 88.2% | 84.4% | 77.7% | | | 6.1 |

TABLE 26
Calcium L-5-methylfolate assay value at 40° C., RH 75%

| | % assay | % assay | % assay | % assay | |
|---|---|---|---|---|---|
| | value | value | value | value | |
| | T = 30 days | T = 60 days | T = 90 days | T = 180 days | SD |
| Tablet 5 | 81.6% | 72.0% | 71.1% | 67.4% | 2.3 |
| Compar- | | | | | |
| ative | | | | | |
| example | | | | | |
| Tablet 6 | 82.2% | 79.5% | 76.8% | 65.7% | 2.1 |
| Compar- | | | | | |
| ative | | | | | |
| example | | | | | |
| Tablet 7 | 82.0% | 78.6% | 70.9% | 69.9% | 4.8 |
| Tablet 8 | 99.2% | 93.5% | 88.4% | 84.2% | 3.2 |
| Tablet 9 | 100.0% | 100.0% | 97.2% | 88.9% | 4.8 |
| Tablet 10 | 74.2% | 70.2% | 54.3% | 20.6% | 3.3 |
| Tablet 11 | 86.8% | 78.2% | 76.3% | 68.1% | 6.1 |

### Example 19: Effect of the Coadministration of Methyl Folate and L-Acetyl Carnitine in a Validated Animal Model of Human Depression

Forty, 7-week-old male C57Black/6J, mice were subjected to CUS (“Chronic Unpredictable Mild Stress”), a validate model that allow the depressive phenotype as described in J H Cryan et al. in J. Neubiorev. 2005, 03, 009.

The animals were divided in five groups and administered with the following treatments:

Group I: 8 animals, Control, Saline solution

Group II: 8 animals, CUS, Saline Solution

Group III: 8 animals, CUS, methyl folate (MTHF) 3 mg/kg

Group IV: 8 animals; CUS, L-acetyl carnitine (LAC) 30 mg/kg and saline

Group V: 8 animals; CUS, co-administration LAC 30 mg/kg and MTHF 3 mg/kg

Mice were housed 4-5 per cage with free access to food and water in a room at a controlled temperature (21-23° C.) with a light/dark period of 12 hours. The combined antidepressant effect of ACL and MF was evaluated both in animals subjected to CUS, and in non-stressed control animals.

The mice were subjected to CUS for 4 weeks, during which different types of stress were applied to the animals twice-a-day in a random and unpredictable manner: one session during the day lasting 1-3 hours, and one session during the night (lasting 12 hours), with at least 6 hours of interval between the two sessions. The stressful procedures applied are the following: food deprivation; cage placed on a rotating platform; 45° inclined cage; wet litter (250 ml of water at 21° C. for 750 ml of litter; light on during the night; light off during the day; containment inside special transparent Plexiglas cylinders, well ventilated with 0.4 cm openings, 125×5 cm in size, in which the animal can make small back and forth movements but not turn around; change of cage (mice are placed in a cage previously occupied by other mice); strobe light during the night. The CUS animals and the respective controls were subjected to behavioural tests suitable both to evaluate the depressive-like phenotype and the antidepressant effect induced by the subsequent pharmacological treatment.

Drug treatment started at the 3rd week of CUS and it was maintained for 2 weeks, the study ending at Week 5 for the start of the experiment. Behavioural tests were performed 3 days after the beginning of treatments, while post-mortem protein studies in the mouse brain were performed after 14 days of treatment, at the end of Week 5.

To assess the antidepressant behavioural effects of the selected product LAC and MEF the animals were submitted to the Forced swimming test (“FST”) as described in J H Cryan et al. in J. Neubiorev. 2005, 03, 009 and the Total Immobility Time measured in the 5 group of animals.

The FST was previously validated in several articles, the Total Immobility Time being consistently reduced by acute, subacute and chronic treatment with standard antidepressant drugs, such as serotonin-reuptake inhibitors (SSRI). During the test the mice were placed individually for 6 minutes in transparent plexiglass cylinders (25 cm high and 22 cm in diameter) filled with warm water (28° C.), in sufficient quantity to prevent the mouse from touching the bottom of the cylinder with hind legs. The animal was videotaped, and then the immobility time (expressed in seconds) was measured during the last 4 minutes of the test. Table 27 reports the behavioural Antidepressant Test in Mice.

TABLE 27

| | Total Immobility Time |
|---|---|
| Forced swimming test | (sec) + SD |
| Group I (Saline) | 184.87 + 41.47 |
| Group II (CUS-Saline) | 214.25 + 36.83 |
| Group III (MTHF 3 mg/Kg) | 230 + 14.20 |
| Group IV (LAC 30 mg/Kg) | 245.75 + 30.34 |
| Group V (LAC30 + MTHF 3 mg/kg) | 149.5 + 33.93 |

Statistical analysis run with the Anova Fisher's exact test showed significant effect: F (4.35)=10.95

### Example 20: Expression of BDNF, in Limbic Structure of Mice Exposed to Chronic Stress and by Treatments with MTHF and/or LAC

Mice were treated as described in Example 19. After 4 weeks of CUS and 2 week of pharmacological treatments (as described in Example 19), at the end of Week 5 the animals were sacrificed and the level of BDNF protein were measured in Hippocampus and Frontal Cortex by western blot. Table 28 reports the protein levels of BDNF, a neuronal trophic factor related to chronic stress and depression, in the Frontal Cortex of mice.

TABLE 28

| Western blot BDNF in frontal cortex | Optical Density bands + SD |
|---|---|
| Group I (Saline) | 100.64 + 23.05 |
| Group II (CUS-Saline) | 104.70 + 21 |
| Group III (MTHF 3 mg/Kg) | 138.46 + 18.26 |
| Group IV (LAC 30 mg/Kg) | 113.29 + 28.95 |
| Group V(MTHF 3 + LAC 30 mg/Kg) | 203.47 + 72.06 |

For the Western blot for BDNF in frontal cortex the Anova Fisher's exact was: F(6.23)=5.886

FIG. 2 shows the BDNF protein expression levels measured in the mouse I frontal cortex by western blot. The antibody recognizes the mature form of the 17 KDa BDNF. Treatment with the LAC 30 mg with MTHF 3 mg combination induced a significant increase in the intensity of the western blots using specific antibodies to blot BDNF compared to the groups treated with LAC and MTHF separately.

1. A tablet composition comprising granules of crystalline calcium L-methylfolate (MTHF), in an amount from 5 to 40% (w/w) with granules comprising a carnitine derivative salt in an amount from 50 to 90% (w/w), on the weight of the finished composition, together with pharmaceutically acceptable excipients, wherein the methylfolate granules comprise at least an antioxidant or a stabilizing agent.

2. The composition according to claim 1, wherein the antioxidant or the stabilizing agent is selected from ascorbic acid and citric acid in an amount from 3 to 30% (w/w) on the weight of the granules.

3. (canceled)

4. The composition according to claim 1, wherein the methylfolate granules comprise:
2-10% (w/w) of calcium methylfolate;
3-30% (w/w) of antioxidant agent(s) or stabilizer(s);
50-90% (w/w) of diluting agent(s);
4-10% (w/w) of binding agent(s).

5. The composition according to claim 4, wherein the MTHF granules comprise:
2-8% (w/w) of calcium methylfolate;
5-25% (w/w) of antioxidant agent(s) or stabilizer(s);
60-90% (w/w) of diluting agent(s);
3-8% (w/w) of binding agent(s).

6. The composition according to claim 5, wherein the granules comprise:
2-8% (w/w) of calcium methylfolate;
5-25% (w/w) citric acid or ascorbic acid;
60-90% (w/w) corn starch or microcrystalline cellulose;
3-8% (w/w) pregelatinized starch or hydroxypropyl cellulose.

7. The composition according to claim 1, wherein the carnitine derivative salt is acetyl L-carnitine hydrochloride.

8. (canceled)

9. The composition according to claim 7, wherein the acetyl L-carnitine granules comprise acetyl L-carnitine in an amount from 75% to 90% (w/w), polyvinylpyrrolidone in an amount from 3 to 10% (w/w) and microcrystalline cellulose in an amount from 3 to 10% (w/w), on the weight of the granules.

10. The composition according to claim 1, in form of tablet comprising:
5-40% (w/w) of MTHF granules;
50-90% (w/w) of acetyl L-carnitine granules;
0.1-15% (w/w) of lubricants;
0.1-5% (w/w) of glidants;
0-10% (w/w) of diluents;
0-10% (w/w) of vitamins; and
optionally filmed with film-forming coating.

11. The composition according to claim 10, consisting of 590 mg of acetyl L-carnitine HCl, 35 mg of microcrystalline cellulose, 56 mg of polyvinylpyrrolidone, 15 mg of methylfolate calcium salt, 33 mg of ascorbic acid, 172 mg of starch corn, 23 mg of pregelatinized starch, 10 mg of magnesium stearate, 3 mg of colloidal silica and film-forming coating.

12. The composition according to claim 10, consisting of 590 mg of acetyl L-carnitine HCl, 35 mg of microcrystalline cellulose, 56 mg of polyvinylpyrrolidone, 7.5 mg of methylfolate calcium salt, 33 mg of ascorbic acid, 190 mg of starch corn, 23 mg of pregelatinized starch, 10 mg of magnesium stearate, 3 mg of colloidal silica and film-forming coating.

13. The composition according to claim 10, consisting of 590 mg of acetyl L-carnitine HCl, 35 mg of microcrystalline cellulose, 56 mg of polyvinylpyrrolidone, 3 mg of methylfolate calcium salt, 6 mg of ascorbic acid; 120 mg of corn starch; 5 mg of pregelatinized starch, 35 mg of vitamin B6, 2 mg of vitamin B12, 10 mg of magnesium stearate, 3 mg of colloidal silica and film-forming coating.

14.-15. (canceled)

16. A process for the preparation of the composition according to claim 1, comprising the steps of:
a) preparing MTHF granules, wherein MTHF in an amount of 3 to 10% (w/w) I mixed with an amount from 3 to 15% (w/w) of antioxidant or stabilizer, an amount from 50 to 90% (w/w) of diluent and an amount of from 4 to 10% (w/w) of binder, with respect to the weight of the granulate;
b) preparing carnitine granules, wherein acetyl L-carnitine hydrochloride in an amount from 75 to 90% (w/w) is mixed with microcrystalline cellulose in amount from 5 to 10% (w/w) and polyvinylpyrrolidone from 5 to 10% (w/w) with respect to the final weight of the granulate;
c) mixing the MTHF granules obtained in step a) with the carnitine derivative granules obtained in step b) with the extragranular excipients and compressing in tablet form.

17. The process according to claim 16, wherein in step c, the granules obtained in steps a) and b) are mixed with vitamin of group B in an amount from 0 to 10% on the weight of the finished composition.

18. (canceled)

19. A method of treating and/or preventing pathologies connected with depression and/or schizophrenia in individuals in need thereof with the composition according to claim 1, said method comprising
administering to said individuals a pharmaceutical effective amount of said composition.

20. The method according to claim 19, wherein the MTHF dosage is from 5 to 90 mg/day and LAC dosage is from 100 to 1000 mg/day and wherein the composition can be administered one or two time a day, alone or in combination with other antidepressive compounds.

21. A method of treating and/or preventing pathologies connected with neuropathies in individuals in need thereof with the composition according to claim 1, said method comprising
administering to said individuals a pharmaceutical effective amount of said composition.

22. The method according to claim 21, wherein MTHF is in a dosage from 2 to 6 mg, acetyl L-carnitine is in a dosage from 100 mg to 1000 mg with B Group vitamins, wherein said B Group vitamins are vitamin B6 and/or vitamin B12, in an amount ranging from 2 to 50 mg.

23. The composition according to claim 10 comprising from 2 to 30 mg of calcium-L-methylfolate granules and from 400 to 700 mg of acetyl L-carnitine hydrochloride granules.
